(19) United States
(12) Patent Application Publication
(10) Pub. No.: US 2006/0235504 A1
(43) Pub. Date: Oct. 19, 2006

(54) Methods and apparatus for treatment of luminal hyperplasia

(75) Inventor: Gilbert R. Gonzales, New York, NY (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Clear Vascular, Inc., Palo Alto, CA (US)

(21) Appl. No.: 11/351,435

(22) Filed: Feb. 9, 2006

Related U.S. Application Data
(60) Provisional application No. 60/652,129, filed on Feb. 10, 2005.

(51) Int. Cl. A61F 2/90

(52) U.S. Cl. 623/1.15; 128/898

(57) ABSTRACT

A luminal implant comprises a conversion electron emitting source present on an expandable scaffold. Implant may be positioned within a blood vessel or other luminal site at risk of hyperplasia or neoplasia. The conversion electrons emitted by the source will inhibit hyperplasia or neoplasia.

### CROSS-REFERENCES TO RELATED APPLICATIONS

The present application is a non-provisional of U.S. Patent Application Ser. No. 60/652,129 (Attorney Docket No. 025979-000100US), filed Feb. 10, 2005, the full disclosure of which is incorporated herein by reference.

### BACKGROUND OF THE INVENTION

### Field of the Invention

The present invention relates generally to medical devices and methods. More particularly, the present invention relates to luminal prostheses, such as vascular stents and grafts, which have been modified to inhibit restenosis and hyperplasia.

A number of percutaneous intravascular procedures have been developed for treating stenotic atherosclerotic regions of a patient's vasculature to restore adequate blood flow. The most successful of these treatments is percutaneous transluminal angioplasty (PTA). In PTA, a catheter, having an expandable distal end usually in the form of an inflatable balloon, is positioned in the blood vessel at the stenotic site. The expandable end is expanded to dilate the vessel to restore adequate blood flow beyond the diseased region. Other procedures for opening stenotic regions include directional atherectomy, rotational atherectomy, laser angioplasty, stenting, and the like. While these procedures have gained wide acceptance (either alone or in combination, particularly PTA in combination with stenting), they continue to suffer from significant disadvantages. A particularly common disadvantage with PTA and other known procedures for opening stenotic regions is the frequent occurrence of restenosis.

Restenosis refers to the re-narrowing of an artery after an initially successful angioplasty. In the absence of stenting, restenosis afflicts approximately up to 50% of all angioplasty patients and is the result of injury to the blood vessel wall during the lumen opening angioplasty procedure. In some patients, the injury initiates a repair response that is characterized by smooth muscle cell proliferation referred to as “hyperplasia” in the region traumatized by the angioplasty. This proliferation of smooth muscle cells re-narrows the lumen that was opened by the angioplasty within a few weeks to a few months, thereby necessitating a repeat PTA or other procedure to alleviate the restenosis.

A number of strategies have been proposed to treat hyperplasia and reduce restenosis. Previously proposed strategies include prolonged balloon inflation during angioplasty, treatment of the blood vessel with a heated balloon, treatment of the blood vessel with radiation following angioplasty, stenting of the region, and other procedures. While these proposals have enjoyed varying levels of success, no one of these procedures is proven to be entirely successful in substantially or completely avoiding all occurrences of restenosis and hyperplasia.

As an alternative or adjunctive to the above mentioned therapies, the administration of therapeutic agents following PTA for the inhibition of restenosis has also been proposed. Therapeutic treatments usually entail pushing or releasing a drug through a catheter or from a stent. While holding great promise, the delivery of therapeutic agents for the inhibition of restenosis has not been entirely successful.

Of particular interest to the present invention, the use of radiation and “radioactive” stents for inhibiting vascular hyperplasia has been proposed. Radioactive intraluminal endovascular stents prevent neointimal hyperplasia by nonselectively killing dividing cells. The long term consequences of radiation and other stent-delivered therapies on normal heart and on non-cardiac tissue are unknown. Beta particle emitting radioisotopes attached to stents, such as phosphorus-32, deliver 95% of the radiation dose within 4 mm of the stent edge and the radiation dose decreases to less than 1/1000 of the original dose at five months post implantation. The depth or distance into tissue by beta emitters is excessively deep as the ideal inhibition of cell accumulation around the stent should prevent restenosis and yet allow a thin cover to form. The inhibition of proliferation of inflammatory cells in the coronary arteries is required to depths of 100 to 250 micrometers or one tenth to one fourth of a millimeter from the intimal layer edge inward towards the external edge of the coronary artery.

For these reasons, it would be advantageous to provide improved and alternative methods and apparatus for inhibiting hyperplasia in the vasculature following angioplasty and other interventional treatments. It would be further desirable if the improved methods and devices were useful for treating other luminal hyperplasia and neoplasia, including tumors and other neoplastic disease which can occlude and otherwise interfere with the functuality of body lumens. A radioactive stent that is used therapeutically to prevent restenosis and that has the additional benefit of being able to be imaged using a gamma camera would be of clinical utility in determining the position of the stent, to determine radioactive dosimetry and decay and potentially to reveal an internal imaging of soft plaque and hard plaque material. The present invention meets at least some of these objectives.

### BRIEF SUMMARY OF THE INVENTION

The present invention provides improved methods and devices for treating body lumens with radiation for a variety of purposes. In particular, blood vessels and other body lumens are treated by implanting a conversion electron emitting source (CEES) in order to inhibit hyperplasia or neoplasia which might otherwise occur in the body lumen. In the case of blood vessels, particularly arteries, the conversion electron radiation will inhibit hyperplasia and reduce the resulting restenosis which often occurs after angioplasty and other intravascular interventions. As used herein, the term “inhibit” means to reduce hyperplasia by at least 30% relative to the hyperplasia which would occur in the absence of the radiation (“uncontrolled hyperplasia”), typically preventing at least 50% of such hyperplasia, and often preventing 75% or more of the uncontrolled hyperplasia.

The apparatus of the present invention will typically comprise luminal implants which are suitable for implantation at a susceptible site to hyperplasia or neoplasia in the vasculature or other body lumen. Such susceptible sites are typically luminal sites which have been injured, or which may become injured, as a result of disease or trauma. Most typically, the injuries may occur following an interventional procedure such as an intravascular intervention, such as the interventions which are used to treat stenotic, thrombotic, or other conditions in a blood vessel, such as an artery, usually a coronary artery. The most common interventional procedure is balloon angioplasty where a high-pressure balloon is inflated within the coronary artery or other blood vessel in order to widen the lumen of the blood vessel. Other vascular interventions include directional atherectomy, rotational atherectomy, laser angioplasty, stenting, and the like. In addition to blood vessels, the methods and devices of the present invention may be used to treat other body lumens, such as ureters, urethras, hepatic ducts, and other body lumens and ducts which are susceptible to impairment by neoplastic disease.

The luminal implants of the present invention will usually comprise an expandable scaffold which can be delivered to the blood vessel or other body lumen in a collapsed configuration and expanded in situ at a target site within the body lumen. Such expandable scaffolds will usually be in the form of a “stent” or “graft.” Such stents and grafts are well-known and amply described in the patent and medical literature. Typically, the stents and grafts may be either “balloon expandable” where they are formed from a malleable material which is expanded in situ by expansion of a balloon within a lumen of the stent or graft. Alternatively, the stent or graft may be “self-expanding,” typically being formed from a shape-memory alloy other highly elastic material which allows the stent or graft to be constrained during delivery and released from constraint at the target site within the body lumen. In both cases, the stent or graft will normally provide sufficient radially outward or “hoop” strength in order to help hold the blood vessel or other body lumen open in order to maintain patency of the lumen. Such stents and grafts will typically be delivered to blood vessels immediately following angioplasty or other primary interventional treatments.

The conversion electron emitting source (CEES) may be in any form suitable for coupling to the scaffold or other structural components of the luminal implant. In particular, the CEES can be tin-117m, holmium-166, thallium-201, and technicium-99m (but only tin-117m has primarily conversion electron emission).

The use of tin-117m as the CEES is preferred. The tin-117m will usually be in metallic form and can be prepared in an accelerator, such as a cyclotron, by transmutation of antimony into no-carrier-added tin-117 m by high energy proton induced nuclear reactions. Alternatively, fast neutron bombardment, using uranium-235, uranium-233 or plutonium-239, can be accomplished in a reactor.

The preferred metallic tin-117m can be combined with or coupled to the scaffold of a luminal implant in a variety of ways. For example, the tin-117m alloy may be formed directly as a structural component of the scaffold or as the entire scaffold. Alternatively, the tin-117m may be coated or otherwise formed over at least a portion of a scaffold composed of a different material. For example, the tin-117m may be electroplated over the scaffold, may be coated in a carrier over the scaffold, or the like. Still further alternatively, the tin-117m may be deposited into pores of the scaffold or may immobilized within a well or through-hole within the scaffold. As a still further alternative, the tin-117m may be filled within hollow cavities in the structural components of the scaffold.

The scaffold may comprise any conventional stent or graft material, typically being a metal. As noted above, the metal may actually be formed in part or entirely of tin, including tin-117m. The metal could also be stainless steel, platinum, shape-memory alloys, such as nitinol, or the like. The scaffold may also be formed from a polymer, such as a biodegradable polymer, e.g., PLA and PLGA polymers.

Methods according to the present invention for inhibiting hyperplasia in body lumens comprise implanting a source of conversion electrons in the body lumen. The body lumens will typically be at risk of hyperplasia, including blood vessels, ureters, urethras, arteriovenous dialysis shunts, microvascular arteries, arterioles, veins and venules (especially macular and meningorachidian vessels), vaginal canal, cervical os, esophagus, trachea, bronchioles, bronchi, gastrointestinal tract, ostomies, biliary and pancreatic ducts, and the like. The methods of the present invention will find their greatest use, however, in treating arteries for hyperplasia following angioplasty and other primary interventions.

The source of conversion electrons may be any of the CEES's described above, and will typically be formed on a scaffold or other suitable luminal prosthesis as described above. The CEES will be adapted to provide a therapeutically effective radiation emission, typically in the range from 0.0125 mCi/mm to 150 mCi/mm, usually in the range from 0.125 mCi/mm to 7.5 mCi/mm, more usually in the range from 0.125 mCi/mm to 0.75 mCi/mm. Implantation typically comprises expanding the scaffold within the body lumen, and specific CEES materials may be any of those described above.

The half-life (t½) of tin-117m is 14 days and the effective therapeutic time is 28 days or equal to two half-lives. The storage time can be increased by either increasing the purity of the tin-117m/mg or by increasing the electroplating, electrodeposition or other method of adhering the tin-117m to the stent or platform to allow for the radioactive decay. Monthly preparation and distribution of stent batches to cardiovascular use-centers, such as hospitals or local distribution centers, is possible. Each stent batch would have a 3 to 5 day window-of-use differential (quantitatively) of plated stents and this will accomplish adequate availability of stents for use so that shipping of stents can be performed on a monthly or 2 week basis. For example, if a first stent batch has a 5 day usability window from the time of delivery to the cardiovascular use-center until the time the stent must be put into a human coronary artery, this stent would have a set mCi/mm and mCi/mg level of radioactivity placed on it for calender days 1 through 5; for example usability days March 1 through 5. For a second stent batch delivered on the first day of March but for use on days 6 though 10 of the month, the radioactivity level of plating or deposition would be that of first stent batch plus the average decay for 5 days so that on day 6 of March, the stent would have the same radioactivity as the first stent batch on March 1. On March 1 a set of stents for use on March 10 through March 15 would also be delivered but would have plating of levels of radioactivity of tin-117m as that of the first stent batch plus enough tin-117m to compensate for 10 days of decay so that the third stent batch would have the same radioactivity on day 10 of March as the first stent batch has on day 1 of March. The fourth through sixth stent batches would have proportionally larger amounts of tin-117m deposited on them to equal the radioactivity as the first stent batch for use on its first designated and approved day. In this example a total of six batches of stents could be delivered on the first part of each month with each batch implantable for successive five day intervals during the month.

### BRIEF DESCRIPTION OF THE DRAWINGS

FIG. 1 is a perspective view of a luminal prosthesis constructed in accordance with the principles of the present invention.

FIG. 2 is a cross-sectional view of a strut of the prosthesis of FIG. 1, with a CEES

FIG. 3 is a cross-sectional view of a strut of the prosthesis of FIG. 1, with a CEES layer plated over the entire exterior periphery.

FIG. 4 is a cross-sectional view of a strut of the prosthesis of FIG. 1, with a CEES plug in a well.

FIG. 5 is a cross-sectional view of a strut of the prosthesis of FIG. 1, with a CEES plug in a hole.

FIG. 6 is a cross-sectional view of a strut of the prosthesis of FIG. 1, with a CEES impregnated into pores.

FIG. 7 is a cross-sectional view of a strut of the prosthesis of FIG. 1, with a CEES core.

### DETAILED DESCRIPTION OF THE INVENTION

Conversion electron emission is a unique form of radiation with low toxicity and specific physico-chemical properties. The conversion electron is an electron ejected from the atom in the process of internal conversion. Internal conversion is a photophysical process producing an isoenergetic radiationless transition between two electronic states of the same multiplicity. When the transition results in a vibrationally excited molecular entity in the lower electronic state, the entity usually undergoes deactivation to its lowest vibrational level, provided the final stage is not unstable to dissociation.

Tin-117m delivers conversional electrons at a short range and delivers mono-energetic radiation that, in higher concentrations, has significant anti-neoplastic properties. According to the present invention, in small doses, the conversion electrons from tin-117m will produce an anti-inflammatory cellular response to inhibit excess accumulation of cells around and inside an implant, such as a vascular stent. In particular, use of the conversion electron emitting implants is believed to allow for a thin layer of endothelial cell growth on the stent to provide a smooth inner covering to incorporate the device into the artery itself. Such endothelization can help prevent subsequent thrombosis and restenosis of the artery or other blood vessel.

While effective to inhibit hyperplasia and restenosis, the conversion electrons emitted by tin-117m, with its low-energy conversion electron emission and short range in tissue, will not produce significant radiation damage to non-hyperplastic and neoproliferative cells. Such limited energy delivery is believed to prevent the excessive cell growth at either end of a stent which is been observed with the use of prior radiation-emitting stents, commonly referred to as the “candy wrapper” effect. The candy wrapper effect may require different concentrations of CEES along the length of the prosthesis to provide relatively less or in some cases more radioactivity towards the two terminal ends of the stent in comparison to the radioactivity of the rest of the stent, in order to reduce this effect. This differential distribution of radioactivity could also preclude radioactivity exposure to the outer edge of the stent so that only the inner most (i.e., non-outer edge) of the vessel lumen in contact to the stent is exposed to radioactivity. Alternatively, a higher differential distribution of tin-117m radioactivity at the stent ends would produce a higher exposure to the artery beyond the scaffolding effect of the stent and to include areas of the vessel or lumen that may have inflammatory activity but which does not require scaffolding.

The use of tin-117m is particularly preferred since it has a half-life of 14 days with an electron energy of 0.13 MeV and 0.15 MeV with no average due the discrete energy delivery. The depth of conversion electron delivery is thus between 0.22 mm and 0.29 mm which will significantly limit any damage to underlying tissues while providing effective of the arterial wall to inhibit hyperplasia. Twenty-eight days (two half-lives) is believed to be about the optimum period for neoproliferative tissue suppression and suppression of neointimal proliferation following angioplasty and other primary interventional treatments in the coronary arteries and other vasculature.

Preferred implants according to the present invention are metal stents with high specific activity tin-117m, including tin-117m with no carriers added, having a concentration of at least about 21 mCi/mg and a total radiation emission in the range from 0.0125 mCi/mm per stent and up to 150 mCi/mm per stent. Stents having lower specific activities are also contemplated where the tin-117m may be combined with a carrier, typically being deposited, impregnated, electroplated, or otherwise coated on or incorporated within the structure of a metal stent. The tin-117m may also be incorporated into polymeric carriers, such as bioerodable or other plastic stent bodies. In a first example, tin-117m may be incorporated directly into at least a part of a stent made from non-isotopic tin, e.g., by electroplating. The stent body, however, can be made of other metals, including stainless steel and shape-memory alloys, such as nitinol.

The amount of radiation provided by an implant according to the present invention will depend on the purpose of the implant. Implants intended to reduce cell migration and endothelization or to stop all cell activity by destroying cells down to a depth from the stent surface of 0.29 mm will usually have the tin-117m implanted, coated, plated, or otherwise present directly on the surface of the stent to directly contact the wall of the body lumen being treated. If treatment at a lesser depth is desired, the tin-117m can be incorporated within the stent structure to reduce the depth of tissue exposure. For example, a hollow stent filled with the tin-117m can produce a relatively shallow depth of tissue irradiation with higher focused concentration. A thin shelled, hollow core, tubular stent filled with tin-117m will irradiate conversion electrons through the thickness of the shell. The depth of tissue irradiation will be equal to 0.29 mm (i.e. the longest depth of conversion electron emission) minus the thickness of the shell. For example, if the shell is 0.10 mm in thickness and the core is filled with tin-117m, the tissue irradiation depth will be 0.19 mm.

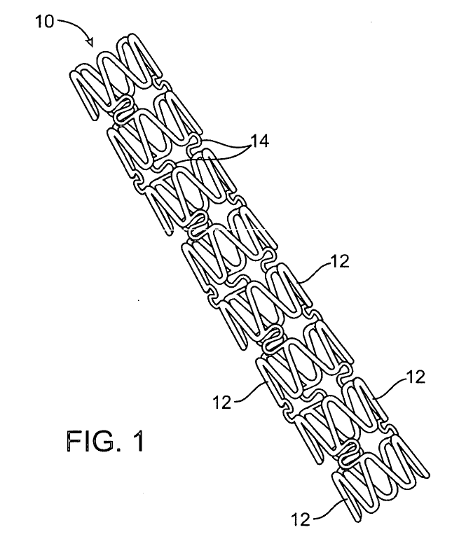

Referring to FIG. 1, an exemplary implant in the form of a vascular stent 10 is illustrated. The stent 10 comprises a plurality of radially expansible segments 12 joined by serpentine connectors 14. The stent 10 is exemplary and is the common type of coronary stent used for treatment following angioplasty or primary cardiac intervention. The stent will be composed of metal, such as stainless steel, tin, cobalt chromium, or a shape-memory alloy such as nickel titanium alloy, and will have tin-117m incorporated in its structure in any one of the ways described above.

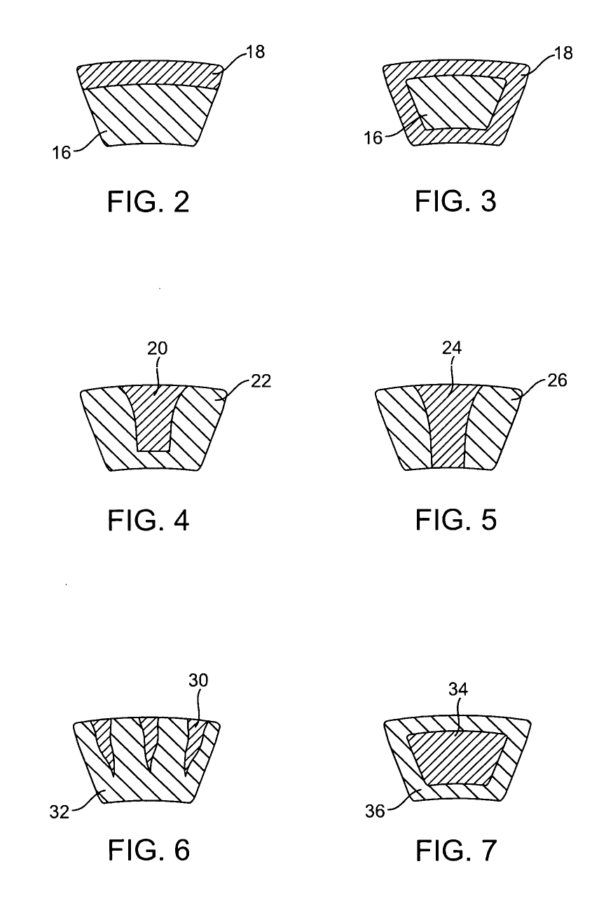

In particular, as shown in FIG. 2, a metal component 16 of the stent may have the tin-117m or other CEES material 18 plated over an exterior surface of the component. Plating of 316 stainless steel stents may be performed by applying a cobalt nuclectron layer followed by a HCl and COCl₂ bath with current exposure at room temperature to Sn⁺²-117m in an electroplating cell. Alternatively, as shown in FIG. 3, the structural component 16 may have the tin-117m or other CEES layer plated or coated over its entire exterior surface. The CEES may also be deposited in a well 20 of a structural component 22, as shown in FIG. 4, or within a through-hole 24 of a structural component 26, as shown in FIG. 5. Other approaches for incorporating the tin-117m include filling pores or other interstices 30 within the structural component of the stent 32, as shown in FIG. 6 and filling a hollow cavity 34 of the structural component 36 of the stent, as shown in FIG. 7

In all cases, the stent 10 may be delivered to a target artery or other blood vessel or body lumen in a conventional way. For balloon-expandable stents, the stent will be delivered over a delivery of balloon and expanded in situ at the target site. For self-expanding stents, the stent will be delivered while radially constrained, e.g., by an outer sheath, until it is released from constraint in situ at the target luminal site. Such delivery procedures are conventional and well-described in the patent and medical literature.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A luminal implant comprising a conversion electron emitting source (CEES).

2. A luminal implant as in claim 1, comprising an expandable scaffold structure.

3. A luminal implant as in claim 2, wherein the CEES is selected from the group consisting of tin-117m, holmium-166, thallium-201, and technicium-99m.

4. A luminal implant as in claim 3, wherein the source of conversion electrons comprises tin-117m.

5. A luminal implant as in claim 4, wherein at least a portion of the scaffold is composed of tin-117m.

6. A luminal implant as in claim 5, wherein substantially the entire scaffold structure is formed from tin-117m.

7. A luminal implant as in claim 4, wherein the tin-117m is coated over at least a portion of the scaffold.

8. A luminal implant as in claim 4, wherein the tin-117m is electroplated over the scaffold.

9. A luminal implant as in claim 4, wherein the tin-117m is present in a carrier over the scaffold.

10. A luminal implant as in claim 4, wherein the tin-117m is deposited in pores of the scaffold.

11. A luminal implant as in claim 4, wherein the tin-117m is present in wells of the scaffold.

12. A luminal implant as in claim 4, wherein the tin-117m is filled within hollow cavities in the scaffold.

13. A luminal implant as in claim 2, wherein the scaffold comprises a metal.

14. A luminal implant as in claim 13, wherein the metal comprises tin.

15. A luminal implant as in claim 13, wherein the metal is a shape-memory alloy.

16. A luminal prosthesis as in claim 2, wherein the scaffold comprises a polymer.

17. A luminal prosthesis as in claim 16, wherein the polymer is biodegradable.

18. A luminal prosthesis as in any one of claim 1, wherein the implant is a vascular stent.

19. A luminal prosthesis as in any one of claims 1 to 18, wherein the CEES has a uniform concentration along the length of the prosthesis.

20. A luminal prosthesis as in any one of claims 1 to 18, wherein the CEES has a non-uniform concentration along the length of the prosthesis.

21. A luminal prosthesis as in claim 20, wherein the CEES has a higher concentration at at least one end of the prosthesis.

22. A luminal prosthesis as in claim 20, wherein the CEES has a lower concentration at at least one end of the prosthesis.

23. A method for inhibiting hyperplasia in a body lumen, said method comprising:
implanting a source of conversion electrons in a body lumen at risk of hyperplasia.

24. A method as in claim 23, wherein the body lumen is a blood vessel.

25. A method as in claim 24, wherein the blood vessel is an artery.

26. A method as in claim 25, wherein the artery is a coronary artery.

27. A method as in any one of claims 23 to 26, wherein the conversion electrons provide a radiation emission in the range from 0.0125 mCi/mm to 150 mCi/mm.

28. A method as in claim 27, wherein the range is from 0.125 mCi/mm to 0.75 mCi/mm.

29. A method as in any one of claims 23, wherein implanting comprises expanding a scaffold within the body lumen, wherein the scaffold carries a conversion electron emitting source (CEES).

30. A method as in claim 29, wherein the CEES is selected from the group consisting of tin-117m.

31. A method as in claim 30, wherein the CEES is tin-117m.

32. A method as in claim 23, further comprising imaging the implant by detecting photon release in the 0.159 MeV range.
